SEQUENCE LISTING (32 sequences)

SEQ ID NO: 1 - protein, 10 aa, synthetic construct
GFTFSSYTMS

SEQ ID NO: 2 - protein, 17 aa, unidentified
TISGGGANIYYPDSVKG

SEQ ID NO: 3 - protein, 7 aa, synthetic construct
PYYAIDF

SEQ ID NO: 4 - protein, 11 aa, synthetic construct
KASQDVGSAVA

SEQ ID NO: 5 - protein, 7 aa, synthetic construct
WASTRHT

SEQ ID NO: 6 - protein, 9 aa, synthetic construct
QQYSTYTWT

SEQ ID NO: 7 - protein, 116 aa, synthetic construct
EVKLVESGGGLVKPGGSLKLSCAAAGFTFSSYTMSWVRQTPAKRLEWVATISGGGANIYYPDSVKGRFTISRDNARNTLYLQMSSLRSEDTAMYYCVSPYYAIDFWGQGTSVTVSS

SEQ ID NO: 8 - protein, 107 aa, synthetic construct
DTVMTQSDKFMSTSVGDRVSITCKASQDVGSAVAWYQQKPGQSPKLLIYWASTRHTGVPDRFTGSGSGTDFTLTISNVQSEDLADYFCQQYSTYTWTFGGGTKLEIK

SEQ ID NO: 9 - protein, 69 aa, synthetic construct
TTTPAPRPPTPAPTIASQPLSLRPEACRPAAGGAVHTRGLDFACDIYIWAPLAGTCGVLLLSLVITLYC

SEQ ID NO: 10 - protein, 42 aa, synthetic construct
KRGRKKLLYIFKQPFMRPVQTTQEEDGCSCRFPEEEEGGCEL

SEQ ID NO: 11 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 116 aa, synthetic construct
EVQLVESGGGLVQPGGSLRLSCKASGFTFSSYTMSWVRQAPGKGLEWVATISGGGANIYYPDSVKGRFTISRDDSKNTLYLQMNSLRAEDTAVYYCARPYYAIDFWGQGTLVTVSS

SEQ ID NO: 13 - protein, 116 aa, synthetic construct
EVQLVESGGGLVQPGGSLRLSCKASGFTFSSYTMSWVRQAPGKGLEWVATISGGGANIYYPDSVKGRFTISRDNSKNTLYLQMNSLRAEDTAVYYCVSPYYAIDFWGQGTLVTVSS

SEQ ID NO: 14 - protein, 116 aa, synthetic construct
EVQLVESGGGLVQPGGSLRLSCAASGFTFSSYTMSWVRQAPGKGLEWVATISGGGANIYYPDSVKGRFTISRDNSKNTLYLQMNSLRAEDTAVYYCVSPYYAIDFWGQGTLVTVSS

SEQ ID NO: 15 - protein, 107 aa, synthetic construct
DIQMTQSPSSLSASVGDRVTITCKASQDVGSAVAWYQQKPGKAPKLLIYWASTRHTGVPSRFSGSGSGTDFTLTISSLQPEDFATYYCQQYSTYTWTFGQGTKVEIK

SEQ ID NO: 16 - DNA, 348 bp, synthetic construct
  source 1..348: mol_type = other DNA
gaagtgaagctggtggaatctggcggcggactggttaagcctggcggatctctgaagctgtcttgtgccgccgctggcttcaccttcagcagctacacaatgagctgggtccgacagacccctgccaagagactggaatgggtcgccacaatttctggcggaggcgccaacatctactaccccgattctgtgaagggcagattcaccatcagccgggacaacgccagaaacaccctgtacctgcagatgagcagcctgagaagcgaggacaccgccatgtactactgtgtgtctccctactacgccatcgatttctggggccagggcaccagcgtgacagttagtagc

SEQ ID NO: 17 - DNA, 321 bp, synthetic construct
  source 1..321: mol_type = other DNA
gatacagtgatgacccagagcgacaagttcatgagcacctccgtgggcgacagagtgtccatcacctgtaaagccagccaggatgtgggatctgccgtggcctggtatcagcagaagcctggccagtctcctaagctgctgatctactgggccagcaccagacataccggcgtgcccgatagattcacaggctctggcagcggcaccgacttcaccctgacaatcagcaacgtgcagagcgaggacctggccgactacttctgtcagcagtacagcacctacacctggacctttggaggcggcaccaagctggaaatcaag

SEQ ID NO: 18 - DNA, 348 bp, synthetic construct
  source 1..348: mol_type = other DNA
gaggtgcagctggttgaatctggcggaggattggttcagcctggcggctctctgagactgtcctgcaaggcttccggcttcaccttctccagctacaccatgtcctgggtccgacaggctcctggcaaaggactggaatgggtcgccacaatttctggcggcggagccaacatctactaccccgactctgtgaagggcagattcaccatctctcgggacgactccaagaacaccctgtacctgcagatgaactccctgagagccgaggacaccgccgtgtactactgtgccagaccttactacgccatcgatttctggggccagggcaccctggttaccgtttcttct

SEQ ID NO: 19 - DNA, 348 bp, synthetic construct
  source 1..348: mol_type = other DNA
gaagtgcagctggttgaatctggcggaggactggttcagcctggcggatctctgagactgtcctgcaaggcctccggcttcaccttctccagctacaccatgtcctgggtccgacaggctcctggcaaaggactggaatgggtcgccacaatttctggcggcggagccaacatctactaccccgactctgtgaagggcagattcaccatcagccgggacaactccaagaacaccctgtacctgcagatgaactccctgagagccgaggacaccgccgtgtactactgtgtgtccccttactacgccatcgatttctggggccagggcaccctggttaccgtttcttct

SEQ ID NO: 20 - DNA, 348 bp, synthetic construct
  source 1..348: mol_type = other DNA
gaagtgcagctggttgaatctggcggaggactggttcagcctggcggatctctgagactgtcctgcgctgcctccggcttcaccttctccagctacaccatgtcctgggtccgacaggctcctggcaaaggactggaatgggtcgccacaatttctggcggcggagccaacatctactaccccgactctgtgaagggcagattcaccatcagccgggacaactccaagaacaccctgtacctgcagatgaactccctgagagccgaggacaccgccgtgtactactgtgtgtccccttactacgccatcgatttctggggccagggcaccctggttaccgtttcttct

SEQ ID NO: 21 - DNA, 321 bp, synthetic construct
  source 1..321: mol_type = other DNA
gacatccagatgacccagtctccatccagcctgtctgcctctgtgggcgacagagtgaccatcacatgcaaggcctctcaggacgtgggctctgccgttgcttggtatcagcagaagcctggcaaggcccctaagctgctgatctactgggcctccaccagacataccggcgtgccctctagattctccggctctggctctggcaccgactttaccctgacaatctccagcctgcagcctgaggacttcgccacctactactgccagcagtactccacctacacctggacctttggccagggcaccaaggtggaaatcaag

SEQ ID NO: 22 - protein, 18 aa, synthetic construct
EGRGSLLTCGDVEENPGP

SEQ ID NO: 23 - protein, 19 aa, synthetic construct
ATNFSLLKQAGDVEENPGP

SEQ ID NO: 24 - protein, 20 aa, synthetic construct
QCTNYALLKLAGDVESNPGP

SEQ ID NO: 25 - protein, 22 aa, synthetic construct
VKQTLNFDLLKLAGDVESNPGP

SEQ ID NO: 26 - protein, 15 aa, synthetic construct
GGGGSGGGGSGGGGS

SEQ ID NO: 27 - DNA, 207 bp, synthetic construct
  source 1..207: mol_type = other DNA
acgacaaccccagctcctaggccaccaacaccagctccaaccatcgcttcccagccactgtctctgaggccagaggcttgtagacctgctgctggaggagctgtgcacaccaggggactggactttgcctgcgatatctatatctgggctccactggctggaacatgtggcgtgctgctgctgagcctggtcatcaccctgtattgc

SEQ ID NO: 28 - DNA, 126 bp, synthetic construct
  source 1..126: mol_type = other DNA
aagaggggcagaaagaagctgctgtacatcttcaagcagccttttatgaggccagtgcagacaacccaggaggaggacggctgctcctgtagattcccagaggaggaggagggaggatgtgagctg

SEQ ID NO: 29 - DNA, 336 bp, synthetic construct
  source 1..336: mol_type = other DNA
agggtgaagttttctcggagcgccgatgctcccgcctatcagcagggccagaaccagctgtacaacgagctgaatctgggccggagggaggagtacgacgtgctggataagagaaggggacgggaccccgagatgggaggcaagccacggaggaagaacccccaggagggcctgtataatgagctgcagaaggacaagatggctgaggcctactccgagatcggcatgaagggagagagacgccggggcaagggacacgatggcctgtatcagggcctgtctacagccaccaaggacacctacgatgctctgcacatgcaggccctgcctccaaga

SEQ ID NO: 30 - DNA, 63 bp, synthetic construct
  source 1..63: mol_type = other DNA
atggccttaccagtgaccgccttgctcctgccgctggccttgctgctccacgccgccaggccg

SEQ ID NO: 31 - protein, 223 aa, synthetic construct
AASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKR

SEQ ID NO: 32 - DNA, 735 bp, synthetic construct
  source 1..735: mol_type = other DNA
gaggtgcagctggtggaatctggcggaggacttgttcagcctggcggctctctgagactgtcttgtgccgccagcggcttcaccttcagcagctttggaatgcactgggtccgacaggcccctggcaaaggacttgagtgggtcgcctacatcagcagcgatagcagcgccatctactacgccgacaccgtgaagggcagattcaccatctccagagacaacgccaagaacagcctgtacctgcagatgaactccctgcgcgacgaggataccgccgtgtactattgtggcagaggcagagagaacatctattacggcagcagactggactactggggccagggaacaaccgtgacagtctcttctggcggcggaggaagcggaggcggaggctccggcggtggtggatctgatattcagctgactcagagccccagcttcctgagcgcctctgtgggagacagagtgaccatcacatgcaaggccagccagaacgtggacaccaacgtggcctggtatcagcaaaagcctggcaaggcccctaaggctctgatctacagcgccagctacagatacagcggcgtgcccagcagattttctggcagcggctctggcaccgacttcaccctgacaatctccagcctgcagcccgaggacttcgccacctactactgccagcagtacaacaactaccctttcaccttcggccagggcaccaagctggaaatcaagcgt

(19) United States
(12) Patent Application Publication
(10) Pub. No.: US 2025/0381225 A1
(43) Pub. Date: Dec. 18, 2025

(54) RECOMBINANT ANTIBODIES, CHIMERIC ANTIGEN RECEPTORS, AND USES THEREOF IN TREATING CANCERS

(71) Applicant: Development Center for Biotechnology, New Taipei City (TW)

(72) Inventors: Li-Shuang AI, Taipei City (TW); Yu-Hsun LO, Taipei City (TW); Cheng-Chou YU, Taipei City (TW); Yi-Jiue TSAI, Taipei City (TW); Hsin-Yi TSAI, Taipei City (TW); Show-Shan SHEU, Taipei City (TW); Chia-Tsen LAI, Taipei City (TW); Pei-Yi TSAI, Taipei City (TW); Szu-Liang LAI, Taipei City (TW)

(21) Appl. No.: 19/237,136

(22) Filed: Jun. 13, 2025

Related U.S. Application Data
(60) Provisional application No. 63/659,899, filed on Jun. 14, 2024.

(51) Int. Cl. A61K 35/17; A61K 40/11; A61K 40/15; A61K 40/17; A61K 40/31; A61K 40/42; A61P 35/00; C07K 14/705; C07K 14/725; C07K 16/28

(52) U.S. Cl. CPC A61K 35/17; A61K 40/11; A61K 40/15; A61K 40/17; A61K 40/31; A61K 40/421; A61P 35/00; C07K 14/7051; C07K 14/70517; C07K 14/70575; C07K 16/2818; C07K 16/2827; A61K 2239/13; A61K 2239/17; A61K 2239/21; A61K 2239/22; A61K 2239/29; C07K 2317/52; C07K 2317/565; C07K 2317/622

(57) ABSTRACT

Disclosed herein are recombinant antibodies specific to programmed death 1 (PD-1), chimeric antigen receptors (CARs) and genetically modified cells configured to express the CARs on their surfaces and secret the recombinant antibodies specific to PD-1. Also disclosed herein is a method of treating cancer by administering the genetically modified cells to a subject afflicted with cancer.

### CROSS-REFERENCE TO RELATED APPLICATIONS

This application claims the benefit of U.S. Provisional Application No. 63/659,899, filed Jun. 14, 2024; the content of the application is incorporated herein by reference in its entirety.

### REFERENCES TO A SEQUENCE LISTING

The present application is being filed along with a Sequence Listing in an electronic format. The Sequence Listing is provided as a file entitled “P4365_SEQ_AF.xml”, created Jun. 4, 2025, which is 33 KB in size. The information in the electronic format of the Sequence Listing is incorporated herein by reference in its entirety.

### BACKGROUND OF THE INVENTION

### 1. Field of the Invention

The present disclosure in general relates to the field of disease treatment. More particularly, the present disclosure relates to a novel antibody specific to programmed death 1 (PD-1), a novel chimeric antigen receptor T (CAR-T) and their uses in the treatment of cancers.

### 2. Description of Related Art

CAR-T therapy is a type of treatment in which a patent's T cells are genetically engineered to express an artificial T cell receptor (TCR) on their surfaces. The artificial TCR is useful in redirecting the engineered T cells to recognize and eliminate target cells (e.g., cancer cells) expressing a specific target antigen (e.g., tumor-associated antigen, TAA). Since 2017, six CAR-T therapies have been approved by the U.S. Food and Drug Administration (FDA), including tisagenlecleucel for B-cell acute lymphoblastic leukemia (ALL) and B-cell non-Hodgkin lymphoma (NHL); axicabtagene ciloleucel for NHL and follicular lymphoma; brexucabtagene autoleucel for mantle cell lymphoma (MCL) and ALL; lisocabtagene maraleucel for NHL; idecabtagene vicleucel for multiple myeloma; and ciltacabtagene autoleucel for multiple myeloma. However, the broad clinical usage of CAR-T technology is still limited due to several factors, such as life-threatening toxicities, limited efficacy against solid tumors (the immunosuppressive tumor microenvironment (TME) and physical tumor barriers usually limit the penetration and infiltration of CAR-T cells), the development of tumor resistance, and antigen escape.

In view of the foregoing, there is a continuing interest in developing a novel secreted PD-1 antibody for use in CAR-T therapy to treat cancers via overcoming TME.

### SUMMARY

The following presents a simplified summary of the disclosure to provide a basic understanding to the reader. This summary is not an extensive overview of the disclosure, and it does not identify key/critical elements of the present invention or delineate the scope of the present invention. Its sole purpose is to present some concepts disclosed herein in a simplified form as a prelude to the more detailed description that is presented later.

As embodied and broadly described herein, the first aspect of the disclosure is directed to a recombinant antibody. According to some embodiments of the present disclosure, the recombinant antibody is specific to programmed cell death protein 1 (PD-1). The recombinant antibody in its structure comprises a heavy chain variable (VH) domain and a light chain variable (VL) domain, in which the VH domain comprises a first heavy chain complementarity determining region (CDR-H1), a second heavy chain CDR (CDR-H2), and a third heavy chain CDR (CDR-H3); and the VL domain comprises a first light chain CDR (CDR-L1), a second light chain CDR (CDR-L2), and a third light chain CDR (CDR-L3).

According to certain embodiments of the present disclosure, the CDR-H1, CDR-H2, and CDR-H3 of the recombinant antibody respectively comprise the amino acid sequences of “GFTFSSYTMS” (SEQ ID NO: 1), “TISGGGANIYYPDSVKG” (SEQ ID NO: 2), and “PYYAIDF” (SEQ ID NO: 3); and the CDR-L1, CDR-L2, and CDR-L3 of the recombinant antibody respectively comprise the amino acid sequences of “KASQDVGSAVA” (SEQ ID NO: 4), “WASTRHT” (SEQ ID NO: 5), and “QQYSTYTWT” (SEQ ID NO: 6).

In various embodiments of the present disclosure, the VH domain and VL domain of the recombinant antibody respectively comprise the amino acid sequences at least 85% identical to SEQ ID NOs: 7 and 8. According to certain preferred embodiments, the VH domain comprises the amino acid sequence of SEQ ID NO: 7, 12, 13 or 14 (i.e., comprising the amino acid sequence 100% identical to SEQ ID NO: 7, 12, 13 or 14); and the VL domain comprises the amino acid sequence of SEQ ID NO: 8 or 15 (i.e., comprising the amino acid sequence 100% identical to SEQ ID NO: 8 or 15).

According to certain exemplary embodiments, the present recombinant antibody is in the form of a single-chain variable fragment (scFv). Optionally or in addition, the scFv further includes a fragment crystallizable region (Fc region) of an immunoglobulin (e.g., IgG).

According to embodiments of the present disclosure, the recombinant antibody is secreted by an immune cell or a stem cell. Preferably, the immune cell is a T-cell.

Also disclosed herein is an isolated cell (e.g., a T cell) configure to express a chimeric antigen receptor (CAR) and the present recombinant antibody described above. The isolated cell is transformed by a nucleic acid comprising in sequence, from 5′-end to 3′-end,
a first coding sequence encoding a first single-chain variable fragment (scFv) specific to an antigen;
a second coding sequence encoding a hinge and transmembrane (HTM) domain of a first protein;
a third coding sequence encoding a co-stimulatory molecule;
a fourth coding sequence encoding a cytoplasmic domain of a second protein;
an internal ribosomal entry site (IRES) or a linker sequence encoding a 2A peptide; and
a fifth coding sequence encoding a second scFv comprising the present recombinant antibody;
   wherein,
the first to fourth coding sequences collectively encode the CAR, which is disposed on the membrane of the isolated cell after expression; and
the recombinant antibody is secreted out from the isolated cell after expression

According to embodiments of the present disclosure, the first scFv encoded by the first coding sequence is an antigen specific to a TAA; the HTM domain encoded by the second coding sequence is the HTM domain of cluster of differentiation 8 (CD8); the co-stimulatory molecule encoded by the third coding sequence is 4-1BB; and the cytoplasmic domain encoded by the fourth coding sequence is the cytoplasmic domain of CD3 zeta chain (CD32). In some exemplary examples, the HTM domain of CD8 comprises the amino acid sequence of SEQ ID NO: 9; the 4-1BB co-stimulatory molecule comprises the amino acid sequence of SEQ ID NO: 10; and the cytoplasmic domain of CD3 comprises the amino acid sequence of SEQ ID NO: 11.

According to embodiments of the present disclosure, the CDR-H1, CDR-H2, and CDR-H3 of the second scFv encoded by the fifth coding sequence respectively comprise the amino acid sequences of “GFTFSSYTMS” (SEQ ID NO: 1), “TISGGGANIYYPDSVKG” (SEQ ID NO: 2), and “PYYAIDF” (SEQ ID NO: 3); and the CDR-L1, CDR-L2, and CDR-L3 of the second scFv respectively comprise the amino acid sequences of “KASQDVGSAVA” (SEQ ID NO: 4), “WASTRHT” (SEQ ID NO: 5), and “QQYSTYTWT” (SEQ ID NO: 6). According to certain exemplary embodiments, the VH domain and VL domain of the second scFv respectively comprise the amino acid sequences at least 85% identical to SEQ ID NOs: 7 and 8. According to some examples, the VH domain of the second scFv comprises the amino acid sequence of SEQ ID NO: 7, 12, 13 or 14; and the VL domain of the second scFv comprises the amino acid sequence of SEQ ID NO: 8 or 15.

Optionally, in addition to the first, second, third, fourth and fifth coding sequences and the linker sequence, the nucleic acid further comprises a sixth coding sequence linked to the 3′ end of the fifth coding sequence. In these embodiments, the sixth coding sequence encodes a fragment crystallizable region (Fc region) of an immunoglobulin (e.g., IgG). Depending on desired purpose, the immunoglobulin may be an immunoglobulin G (IgG), immunoglobulin A (IgA), immunoglobulin M (IgM), immunoglobulin D (IgD) or immunoglobulin E (IgE). According to one exemplary embodiment, the immunoglobulin is IgG, for example, IgG1 or IgG4.

According to embodiments of the present disclosure, the isolated cell is transformed by an expression vector comprising the nucleic acid described above. The expression vector may be is a viral vector; for example, a lentiviral vector, an adenoviral vector, a retroviral vector, an adeno-associated viral vector, or a sindbis viral vector. In one exemplary embodiment, the expression vector is the lentiviral vector. After transformation, the isolated cell could express the CAR on its surface and secret the present recombinant antibody out of the cell.

Another aspect of the present disclosure thus pertains to the use of a genetically modified cell (i.e., an isolated cell transformed by the present nucleic acid described above) in the treatment of cancers.

According to some embodiments of the present disclosure, the genetically modified cell comprises the nucleic acid described above, thus is configured to express the CAR and the present recombinant antibody, with the CAR being disposed on the membrane of the genetically modified cell while the present recombinant antibody being secreted out of the genetically modified cell after expression. Preferably, the genetically modified cell is a genetically modified immune cell, such as a genetically modified T cell, a genetically modified natural killer (NK) cell, or a genetically modified macrophage.

The genetically modified immune cell is useful in treating cancers via recognizing and specifically binding to the cancers through the CAR. Accordingly, also disclosed herein is a method of treating cancer in a subject. The method comprises administering to the subject an effective amount of the genetically modified immune cell to alleviate or ameliorate the symptoms of the cancer.

Depending on the intended purpose, the cancer may be gastric cancer, lung cancer, bladder cancer, breast cancer, pancreatic cancer, renal cancer, colorectal cancer, cervical cancer, ovarian cancer, brain tumor, prostate cancer, hepatocellular carcinoma, melanoma, esophageal carcinoma, multiple myeloma, or head and neck squamous cell carcinoma.

The subject treatable with the genetically modified immune cell and/or method of the present disclosure is a mammal; preferably, a human.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings briefly discussed below:

FIG. 1 is a schematic diagram depicting a nucleic acid construct encoding a CAR and the present recombinant antibody according to embodiments of the present disclosure;

FIG. 2 are schematic diagrams of specified nucleic acid constructs according to the embodiments of the present disclosure;

FIG. 3 is a line graph depicting the bindings of chimeric anti-human PD-1 2B6 antibody to PD-1 using ELISA according to one embodiment of the present disclosure;

FIG. 4 illustrates the ability of chimeric anti-human PD-1 2B6 antibody to induce PD-1/PD-L1 blockade according to one embodiment of the present disclosure;

FIG. 5 depicts the in vivo efficacy of chimeric anti-human PD-1 2B6 antibody treatment in the murine syngeneic MC38 colon cancer model according to one embodiment of the present disclosure;

FIG. 6 depicts the anti-tumor effect of specified CAR-T cells according to Example 3 of the present disclosure, GH: Globo H CAR-T cell. GH/Nivo: Globo H CAR/Nivo scFv-Fc CAR-T cell. GH/PD-1 2B6-HuB1: Globo H/PD-1^{2B6} scFv-Fc CAR-T cell.

FIGS. 7A to 7D respectively depict the therapeutic effects of specified CAR-T cells on cancers according to Example 4 of the present disclosure, GH CAR-T: Globo H CAR-T cell. GH/Nivo CAR-T: Globo H CAR/Nivo scFv-Fc CAR-T cell. GH/PD-1 2B6-HuB1 CAR-T: Globo H/PD-1^{2B6} scFv-Fc CAR-T cell.

FIG. 8 depicts the therapeutic effects of specified B7H3 CAR-T cells and B7H3/PD-1 2B6HuB1 CAR-T cells on cancers according to Example 5 of present disclosure.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The term “nucleic acid” refers to a polynucleotide such as deoxyribonucleic acid (DNA) and where appropriate, ribonucleic acid (RNA). Nucleic acids include but are not limited to single-stranded and double-stranded polynucleotides. Illustrative polynucleotides include DNA, single-stranded DNA, cDNA, and mRNA. The term also includes, analogs of either DNA or RNA made from nucleotide analogs, and as applicable, single (sense or antisense) and double-stranded polynucleotides. The term further includes modified polynucleotides, including modified DNA and modified RNA, e.g., DNA and RNA comprising one or more unnatural nucleotide or nucleoside. The terms “nucleic acid” is used herein to refer to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, and/or which have similar binding properties as the reference nucleic acid, and/or which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

As used herein, the term “recombinant antibody” refers to an antibody that is expressed and isolated from a cell or cell line transfected with an expression vector (or possibly more than one expression vector, typically two expression vectors) comprising the coding sequence of the antibody, where said coding sequence is not naturally associated with the cell.

The term “antibody” (Ab) is used in its meaning known in the art of cell biology and biochemistry, and covers monoclonal antibodies (including full length monoclonal antibodies), polyclonal antibodies, multi-specific or multivalent antibodies (e.g., bi-specific antibodies), chimeric antibodies, humanized antibodies and antibody fragments so long as they exhibit the desired biological activity. The term “antibody fragment” may be produced by recombinant DNA techniques or by enzymatic or chemical cleavage of intact antibodies. Examples of the antibody fragment include, fragment antigen-binding (Fab), Fab′, F(ab′)2, single-chain variable fragment (scFv), domain antibody (dAb), diabodies, triabodies, tetrabodies, and polypeptides that contain at least a portion of an immunoglobulin that is sufficient to confer specific antigen binding to the polypeptide. According to certain embodiments of the present disclosure, the antibody of the present disclosure is present in the form of a scFv.

The term “single-chain variable fragment” (scFv) is used in its meaning known in the art of cell biology and biochemistry, and refers to a fusion protein of the variable domains of the heavy chain (VH) and light chain (VL) of an immunoglobulin, linked together with a short (usually serine and/or glycine) linker peptide. The scFv retains the specificity of the original immunoglobulin, despite removal of the constant domains and the introduction of the linker.

The term “complementarity determining region” (CDR) used herein refers to the hypervariable region of an antibody molecule that forms a surface complementary to the three-dimensional surface of a bound antigen. Proceeding from N-terminus to C-terminus, each of the antibody heavy and light chains comprises three CDRs (CDR-1, CDR-2 and CDR-3). An antigen combining site, therefore, includes a total of six CDRs that comprise three CDRs in the variable domain of a heavy chain (i.e., CDR-H1, CDR-H2 and CDR-H3), and three CDRs in the variable domain of a light chain (i.e., CDR-L1, CDR-L2 and CDR-L3).

The “variable domain” of an antibody refers to the amino-terminal domains of heavy or light chain of the antibody. These domains are generally the most variable parts of an antibody and contain the antigen-binding sites. The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen-binding site of antibodies.

As discussed herein, minor variations in the amino acid sequences of antibodies (especially minor variations in the FR sequences of antibodies), or in the nucleotide sequences of nucleic acids are contemplated as being encompassed by the presently disclosed and claimed inventive concept(s), providing that the variations in the amino acid sequence/nucleotide sequence maintain at least 85% sequence identity, such as at least 85%, 86%, 87%, 88%, 89%, 90%, 91%, 92%, 93%, 94%, 95%, 96%, 97%, 98% and 99% sequence identity. The antibody of the present disclosure may be modified specifically to alter a feature of the antibody unrelated to its physiological activity. For example, certain amino acid residues in the framework (FR) region of the antibody can be changed and/or deleted without affecting the physiological activity of the antibody in this study (i.e., its ability to treat cancers). In particular, conservative amino acid replacements are contemplated. Conservative replacements are those that take place within a family of amino acid residues that are related in their side chains. Genetically encoded amino acid residues are generally divided into families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid residue within the antigen-biding sites, i.e., CDRs. Whether an amino acid change results in a functional peptide can readily be determined by assaying the specific activity of the peptide derivative. Fragments or analogs of proteins/peptides can be readily prepared by those of ordinary skill in the art. Preferred amino- and carboxy-termini of fragments or analogs occur near boundaries of functional domains.

“Percentage (%) sequence identity” is defined as the percentage of amino acid residues/nucleotides in a candidate sequence that are identical with the amino acid residues/nucleotides in the specific polypeptide/polynucleotide sequence, after aligning the sequences and introducing gaps, if necessary, to achieve the maximum percent sequence identity, and not considering any conservative substitutions as part of the sequence identity. Alignment for purposes of determining percentage sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, sequence comparison between two amino acid sequences/nucleotide sequences was carried out by computer program Blastp (protein-protein BLAST)/Blastn (nucleotide-nucleotide BLAST) provided online by Nation Center for Biotechnology Information (NCBI). The percentage amino acid sequence/nucleotide sequence identity of a given amino acid sequence/nucleic acid A to a given amino acid sequence/nucleic acid B (which can alternatively be phrased as a given amino acid sequence/nucleic acid A that has a certain % amino acid sequence/nucleic acid identity to a given amino acid sequence/nucleic acid B) is calculated by the formula as follows:

$$\frac{X}{Y} \times 100 \%$$

where X is the number of amino acid residues/nucleic acids scored as identical matches by the sequence alignment program BLAST in that program's alignment of A and B, and where Y is the total number of amino acid residues/nucleic acids in A or B, whichever is shorter.

As used herein, the term “link” refers to any means of connecting two components either via direct linkage or via indirect linkage between two components.

As used herein, the term “treat,” “treating” and “treatment” are interchangeable, and encompasses partially or completely preventing, ameliorating, mitigating and/or managing a symptom, a secondary disorder or a condition associated with cancers. The term “treating” as used herein refers to application or administration of the CAR-T cells of the present disclosure to a subject, who has a symptom, a secondary disorder or a condition associated with cancers, with the purpose to partially or completely alleviate, ameliorate, relieve, delay onset of, inhibit progression of, reduce severity of, and/or reduce incidence of one or more symptoms, secondary disorders or features associated with cancers. Symptoms, secondary disorders, and/or conditions associated with cancers include, but are not limited to, nausea, vomiting, loss of appetite, constipation, fatigue, muscle weakness, increased thirst, bone pain or broken bones, swelling or lump, blooding, cough, fever, night sweats, coma and pain. Treatment may be administered to a subject who exhibits only early signs of such symptoms, disorder, and/or condition for the purpose of decreasing the risk of developing the symptoms, secondary disorders, and/or conditions associated with cancers. Treatment is generally “effective” if one or more symptoms or clinical markers are reduced as that term is defined herein. Alternatively, a treatment is “effective” if the progression of a symptom, disorder or condition is reduced or halted.

The term “effective amount” as referred to herein designate the quantity of a component which is sufficient to yield a desired response. For therapeutic purposes, the effective amount is also one in which any toxic or detrimental effects of the component are outweighed by the therapeutically beneficial effects. An effective amount of an agent is not required to cure a disease or condition but will provide a treatment for a disease or condition such that the onset of the disease or condition is delayed, hindered or prevented, or the disease or condition symptoms are ameliorated. The effective amount may be divided into one, two, or more doses in a suitable form to be administered at one, two or more times throughout a designated time period. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the structure of the compounds or its derivatives. Effective amount may be expressed, for example, as grams, milligrams or micrograms; as milligrams per kilogram of body weight (mg/Kg); or as cell numbers of body weight (cells/Kg). Persons having ordinary skills could calculate the human equivalent dose (HED) for the medicament (such as the present CAR-T cells) based on the doses determined from animal models. For example, one may follow the guidance for industry published by U.S. Food and Drug Administration (FDA) entitled “Estimating the Maximum Safe Starting Dose in Initial Clinical Trials for Therapeutics in Adult Healthy Volunteers” in estimating a maximum safe dosage for use in human subjects.

The terms “subject” refers to an animal including the human species that is treatable by the CAR-T cells and/or method of the present invention. The term “subject” is intended to refer to both the male and female gender unless one gender is specifically indicated.

### II. Description of the Invention

It is known that PD-1 and its ligand PD-L1 plays a key role in the formation of tumor immunosuppression that facilitates tumor/cancer cells escaping from immune surveillance via inhibiting the activation of immune cells (e.g., T cells) and enhancing the immune tolerance of the tumor/cancer cells. Accordingly, the present disclosure aims at providing a monoclonal antibody exhibiting binding affinity and inhibitory/neutralizing activity towards PD-1, a nucleic acid encoding a CAR and a monoclonal antibody, and a genetically modified cell configured to express the CAR and the monoclonal antibody specific to PD-1. After the nucleic acid is introduced into an immune cell (e.g., T cell), both the CAR and the monoclonal antibody are expressed thereby rendering the immune cell (e.g., CAR-T cell) with a binding specificity and cytotoxicity toward cancer cells and an inhibitory effect on immunosuppressive factor PD-1 present in tumor microenvironment. The inhibitory effect of the monoclonal antibody on PD-1 results in enhanced anti-tumor response of the CAR-T cell in solid tumors. Accordingly, also disclosed herein are the CAR-expressing immune cell (e.g., CAR-T cell), and uses of the cell in treating cancers.

### (i) Monoclonal Antibody

The first aspect of the present disclosure provides an anti-PD-1 monoclonal antibody (mAb) designated as “2B6”. According to some embodiments of the present disclosure, the present mAb 2B6 exhibits a binding affinity and specificity to PD-1 and is useful in blocking the binding of PD-1 to PD-L1 thereby inhibiting the immunosuppressive response induced by the PD-1/PD-1 pathway.

According to some embodiments of the present disclosure, the present mAb 2B6 is produced by conventional immunization method (i.e., immunizing animals with a specific peptide to induce the animal producing peptide-specific Abs). As would be appreciated, the present mAb 2B6 may alternatively be produced by phage-displayed scFv libraries, or recombinant DNA technology (also known as DNA cloning technology; i.e., constructing and transducing a recombinant DNA encoding a specific Ab into a host cell thereby expressing the Ab).

In structure, the mAb 2B6 comprises three CDRs in the VH domain thereof (i.e., CDR-H1, CDR-H2, and CDR-H3), and three CDRs in the VL domain thereof (i.e., CDR-L1, CDR-L2, and CDR-L3). According to some embodiments of the present disclosure, the CDR-H1, CDR-H2, and CDR-H3 of mAb 2B6 respectively comprise the amino acid sequences of “GFTFSSYTMS” (SEQ ID NO: 1), “TISGGGANIYYPDSVKG” (SEQ ID NO: 2), and “PYYAIDF” (SEQ ID NO: 3); and the CDR-L1, CDR-L2, and CDR-L3 of mAb 2B6 respectively comprise the amino acid sequences of “KASQDVGSAVA” (SEQ ID NO: 4), “WASTRHT” (SEQ ID NO: 5), and “QQYSTYTWT” (SEQ ID NO: 6).

As an example, the amino acid sequences of the VH and VL domains of mAb 2B6 are respectively provided as SEQ ID NOs: 7 and 8, described below, in which the CDRs (i.e., the CDR-H1, CDR-H2 and CDR-H3 of VH domain, and the CDR-L1, CDR-L2 and CDR-L3 of VL domain) are marked in bold letters, in sequence.

(VH domain of mAb 2B6)
SEQ ID NO: 7
EVKLVESGGGLVKPGGSLKLSCAAAGFTFSSYTMSWVRQTPAKRLEWVAT
ISGGGANIYYPDSVKGRFTISRDNARNTLYLQMSSLRSEDTAMYYCVSPY
YAIDFWGQGTSVTVSS
(VL domain of mAb 2B6)
SEQ ID NO: 8
DTVMTQSDKFMSTSVGDRVSITCKASQDVGSAVAWYQQKPGQSPKLLIYW
ASTRHTGVPDRFTGSGSGTDFTLTISNVQSEDLADYFCQQYSTYTWTFGG
GTKLEIK

Since the binding affinity and specificity of an antibody are mainly determined by the CDR sequences thereof, as could be appreciated, the framework (FR) sequences of the VH and VL domains may vary (e.g., being substituted by conserved or non-conserved amino acid residues) without affecting the binding affinity and/or specificity of the present antibody. Preferably, the FR sequence is conservatively substituted by one or more suitable amino acid(s) with similar properties; for example, the substitution of leucine (an nonpolar amino acid residue) by isoleucine, alanine, valine, proline, phenylalanine, or tryptophan (another nonpolar amino acid residue); the substitution of aspartate (an acidic amino acid residue) by glutamate (another acidic amino acid residue); or the substitution of lysine (an basic amino acid residue) by arginine or histidine (another basic amino acid residue).

Based on the conservative substitution, a skilled artisan may substitute the amino acid residue(s) of the FR sequences of the VH and VL domains of mAb 2B6 without affecting its activity and/or effect (i.e., binding to PD-1 and/or blocking the binding of PD-1 to PD-L1). Accordingly, the antibody comprising substituted amino acid(s) in its FR sequences of VH and VL domains are intended to be included within the scope of the present disclosure. According to certain embodiments, the VH domain of mAb 2B6 comprises an amino acid sequence at least 85% (i.e., 85%, 86%, 87%, 88%, 89%, 90%, 91%, 92%, 93%, 94%, 95%, 96%, 97%, 98%, 99% or 100%) identical to SEQ ID NO: 7, and the VL domain of mAb 2B6 comprises an amino acid sequence at least 85% (i.e., 85%, 86%, 87%, 88%, 89%, 90%, 91%, 92%, 93%, 94%, 95%, 96%, 97%, 98%, 99% or 100%) identical to SEQ ID NO: 8. According to some preferred embodiments, the VH and VL domains of mAb 2B6 respectively comprise the amino acid sequences at least 90% identical to SEQ ID NOs: 7 and 8. More preferably, the VH and VL domains of mAb 2B6 respectively comprise the amino acid sequences at least 95% identical to SEQ ID NOs: 7 and 8.

According to certain embodiments of the present disclosure, the VH and VL domains of mAb 2B6 is modified to resemble human antibodies so as to minimize the immunogenicity of the antibody in a human subject. Accordingly, the present disclosure further provides different humanized VH and VL sequences, including 2B6 Hd VH (SEQ ID NO: 12), 2B6 HdB1 VH (SEQ ID NO: 13), 2B6 HuB1 VH (SEQ ID NO: 14), and 2B6 Hd VL (SEQ ID NO: 15).

(2B6 Hd VH)
SEQ ID NO: 12
EVQLVESGGGLVQPGGSLRLSCKASGFTFSSYTMSWVRQAPGKGLEWVAT
ISGGGANIYYPDSVKGRFTISRDDSKNTLYLQMNSLRAEDTAVYYCARPY
YAIDFWGQGTLVTVSS
(2B6 HdB1 VH)
SEQ ID NO: 13
EVQLVESGGGLVQPGGSLRLSCKASGFTFSSYTMSWVRQAPGKGLEWVAT
ISGGGANIYYPDSVKGRFTISRDNSKNTLYLQMNSLRAEDTAVYYCVSPY
YAIDFWGQGTLVTVSS
(2B6 HuB1 VH)
SEQ ID NO: 14
EVQLVESGGGLVQPGGSLRLSCAASGFTFSSYTMSWVRQAPGKGLEWVAT
ISGGGANIYYPDSVKGRFTISRDNSKNTLYLQMNSLRAEDTAVYYCVSPY
YAIDFWGQGTLVTVSS
(2B6 Hd VL)
SEQ ID NO: 15
DIQMTQSPSSLSASVGDRVTITCKASQDVGSAVAWYQQKPGKAPKLLIYW
ASTRHTGVPSRFSGSGSGTDFTLTISSLQPEDFATYYCQQYSTYTWTFGQ
GTKVEIK

Depending on intended purpose, the present mAb 2B6 or humanized 2B6 may be produced in the form of a full antibody (e.g., IgG, IgA, IgM, IgD or IgE), or an antibody fragment (e.g., scFv, Fab, Fab′, F(ab′)₂ or diabody). In some exemplary embodiments of the present disclosure, the present mAb is produced in the form of a scFv, i.e., 2B6 scFv.

(ii) the Present Nucleic Acid Encoding a CAR and the Present mAb 2B6 scFv

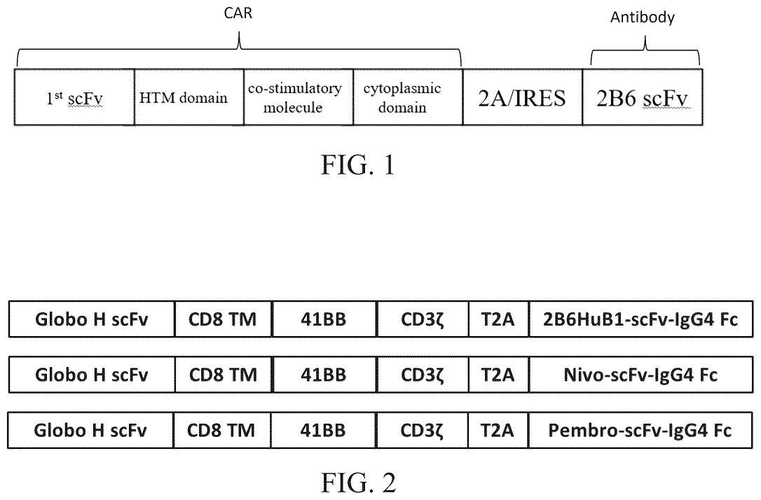

The second aspect of the present disclosure aims at providing a nucleic acid encoding a CAR and the present mAb 2B6 scFv described in Section (i) above. Reference is made to FIG. 1, which is a schematic diagram depicting the nucleic acid construct of the present disclosure. The nucleic acid comprises in sequence, from 5′ end to 3′ end, a first, a second, a third and a fourth coding sequences collectively encode the CAR, an internal ribosomal entry site (IRES) or a linker sequence encoding a 2A peptide, and a fifth coding sequence encoding present mAb 2B6 scFv. Specifically, the first, second, third, fourth and fifth coding sequences respectively encode an antigen (e.g., a first scFv), a hinge and transmembrane (HTM) domain of a first protein (e.g., the HTM domain of CD8), a co-stimulatory molecule (e.g., 4-1BB molecule), a cytoplasmic domain of a second protein (e.g., the cytoplasmic domain of CD3ζ), and the present mAb 2B6 scFv. Note that IRES or the linker sequence encoding 2A peptide is disposed between the CAR encoding sequences and the present mAb coding sequence, allowing the CAR and the present mAb to be expressed independently when the present nucleic acid is translated. Accordingly, the CAR and the present mAb are expressed as two proteins instead of a single fusion protein.

Depending on intended purpose, the antigen or the first scFv may be specific to any antigen expressed/overexpressed and/or associated with tumor/cancer cells, for example, alphafetoprotein (AFP), CA19-9, CA125, carcinoembryonic antigen (CEA), cancer/testis antigen 1B (CTAGIB, also known as “NY-ESO-1”), epithelial tumor antigen (ETA), epidermal growth factor receptor (EGFR), epithelial cell adhesion molecule (EpCAM), folate receptor-alpha (FR-α), human epidermal growth factor receptor-1 (HER1), HER2, HER3, HER4, cell surface-associated mucin 1 (MUC1), melanoma-associated antigen (MAGE), mesothelin (MSLN), prostate-specific membrane antigen (PSMA), prostate stem cell antigen (PSCA), B7 homolog 3 protein (B7-H3), stage-specific embryonic antigen-4 (SSEA-4), tyrosinase, mucin-related Tn, Sialyl Tn, Globo H, ganglioside GD2, CD5, CD19, CD20, CD22, CD23, CD27, CD30, CD33, CD34, CD37, CD38, CD43, CD72a, CD78, CD79a, CD79b, CD86, CD133, CD134, CD137, CD138, or CD319. According to one exemplary example, the first scFv is specific to Globo H. According to one exemplary example, the first scFv is specific to B7-H3.

According to certain embodiments of the present disclosure, the first and second proteins are respectively cluster of differentiation 8 (CD8) and CD3 zeta chain (CD3ζ). Thus, the CD8 HTM domain comprises the amino acid sequence of SEQ ID NO: 9; the 4-1BB co-stimulatory molecule comprises the amino acid sequence of SEQ ID NO: 10; and the cytoplasmic domain of CD3ζ comprises the amino acid sequence of SEQ ID NO: 11. Depending on desired purpose, the hinge domain of the HTM domain of the first protein may alternatively be derived from CD28, IgG1 or IgG4, for example, the hinge domain of CD28, IgG1 or IgG4; and/or the transmembrane domain of the HTM domain of the first protein may alternatively be derived from CD3 zeta chain (CD3ζ), CD8 alpha chain (CD8α), CD4, CD28 or B7-family inducible costimulator (ICOS), for example, the transmembrane domain of CD35, CD8a, CD4, CD28 or ICOS.

As could be appreciated, in addition to the 4-1BB molecule, the present CAR may further comprise other co-stimulatory molecules, such as CD27, CD28 or OX40 (CD134). Alternatively, the 4-1BB molecule of the present CAR may be substituted by other co-stimulatory molecules, such as CD27, CD28 or OX40 (CD134).

The fifth coding sequence encoding the present 2B6 scFv comprises a VH domain-coding fragment and a VL-domain coding fragment. As the name implies, the VH domain-coding fragment encodes the VH domain of the present 2B6 scFv, and the VL-domain coding fragment encodes the VL domain of the present 2B6 scFv. According to certain embodiments of the present disclosure, the fifth coding sequence encodes the mAb 2B6 having the VH domain of SEQ ID NO: 7 and the VL domain of SEQ ID NO: 8; in these embodiments, the VH domain-coding fragment and VL-domain coding fragment of the fifth coding sequence respectively comprise the nucleotide sequences of SEQ ID NOs: 16 and 17. According to some embodiments of the present disclosure, the fifth coding sequence encodes the humanized 2B6 having the VH domain of SEQ ID NO: 12 (2B6 Hd VH) and the VL domain of SEQ ID NO: 15 (2B6 Hd VL); in these embodiments, the VH domain-coding fragment and VL-domain coding fragment of the fifth coding sequence respectively comprise the nucleotide sequences of SEQ ID NOs: 18 and 21. According to further embodiments of the present disclosure, the fifth coding sequence encodes the humanized 2B6 having the VH domain of SEQ ID NO: 13 (2B6 HdB1 VH) and the VL domain of SEQ ID NO: 15 (2B6 Hd VL); in these embodiments, the VH domain-coding fragment and VL-domain coding fragment of the fifth coding sequence respectively comprise the nucleotide sequences of SEQ ID NOs: 19 and 21. According to alternative embodiments of the present disclosure, the fifth coding sequence encodes the humanized 2B6 having the VH domain of SEQ ID NO: 14 (2B6 HuB1 VH) and the VL domain of SEQ ID NO: 15 (2B6 Hd VL); in these embodiments, the VH domain-coding fragment and VL-domain coding fragment of the fifth coding sequence respectively comprise the nucleotide sequences of SEQ ID NOs: 20 and 21.

According to embodiments of the present disclosure, the CAR encoding sequence (i.e., the first to fourth coding sequences) and the second scFv coding sequence are separated by IRES or a linker sequence encoding a 2A peptide. IRES is a sequence that recruit ribosomes and allows cap-independent translation. In practice, IRES serves as a linker linking two coding sequences in one bicistronic vector and allowing the translation of both proteins in cells. 2A peptide also known as “2A self-cleaving peptide” is a class of peptide having 18 to 22 amino acid residues in length, which can induce ribosomal skipping during translation of a protein in cells and help generate polyproteins from a single open reading frame (ORF). In one exemplary embodiment of the present disclosure, the CAR encoding sequence and the 2B6 scFv encoding sequence are linked by the linker sequence that encodes the 2A peptide. Examples of 2A peptide commonly used in the art include, but are not limited to, T2A (EGRGSLLTCGDVEENPGP; SEQ ID NO: 22), P2A (ATNFSLLKQAGDVEENPGP; SEQ ID NO: 23), E2A (QCTNYALLKLAGDVESNPGP; SEQ ID NO: 24) and F2A (VKQTLNFDLLKLAGDVESNPGP; SEQ ID NO: 25). In another exemplary embodiment of the present disclosure, the CAR encoding sequence and the second scFv encoding sequence are linked by IRES.

Optionally or in addition, the second scFv may further comprise a signal peptide (also known as “signal sequence” or “leader sequence”) disposed at its N-terminal. As known in the art, the signal peptide refers to a peptide having about 15-50 amino acid residues in length that directs proteins toward secretory pathway. Examples of the signal peptide suitable to use in the present second scFv include, but are not limited to, the signal peptide of tissue plasminogen activator (tPA), IgK, IgG, CD33, metalloproteinase inhibitor 1 (TIMP1), chronodroitin sulphate proteoglycan 4 (CSPG4), calreticulin (CALR), dickkopf-related protein 3 (DKK3), 60S acidic ribosomal protein P2 (RPLP2), complement C1s (CIS), cathepsin Z (CTSZ), nucleobinin-2 (NUCB2), protein disulphide-isomerase (PDIA1), protein disulphide-isomerase A3 (PDIA3), endoplasmin, hypoxia upregulated protein 1 (HYOU1), trypsinogen-2, serum albumin, and serpinh1. According to one exemplary embodiment of the present disclosure, the signal peptide is derived from light chain of IgG. A skilled artisan may choose a suitable signal peptide in accordance with practical needs.

Preferably, the present second scFv further includes an Fc region of immunoglobulin disposed at its C-terminus (“2B6 scFv-Fc”), the Fc region preferably comprises the glycine (G) and/or serine(S) residues, for example, (G4S) 3 (SEQ ID NO: 26). Accordingly, the present nucleic acid may further include a sixth coding sequence encoding the Fc region of an immunoglobulin (e.g., the Fc region of IgG). Depending on the intended purpose, the immunoglobulin may be IgG, IgA, IgM, IgD or IgE. According to some preferred embodiments, the immunoglobulin is IgG, for example, IgG1, IgG2, IgG3 or IgG4. In some embodiments, the constant region of the immunoglobulin contains a mutation that reduces the binding affinity of the immunoglobulin to an Fc receptor or reduces Fc effector function. For example, the constant region of the immunoglobulin may contain a mutation that eliminates the glycosylation site within the constant region of the heavy chain of immunoglobulin. In some embodiments, the constant region of the immunoglobulin contains one or more mutations, deletions, and/or insertions at an amino acid position corresponding to L234, L235, G236, G237, N297, or P331 of IgG1. In one particular embodiment, the constant region of immunoglobulin contains a mutation at an amino acid position corresponding to N297 of IgG1. In alternative embodiments, the constant region of the immunoglobulin contains one or more mutations, deletions, and/or insertions at an amino acid position corresponding to L281, L282, G283, G284, N344, or P378 of IgG1. In various embodiments of the present disclosure, the immunoglobulin is IgG1 or IgG4.

According to embodiments of the present disclosure, the second, third and fourth coding sequences respectively comprise the nucleotide sequences of SEQ ID NOs: 27, 28 and 29. As could be appreciated, the present second, third and/or fourth coding sequences may be modified to comprise one or more degenerate nucleotides as long as the protein(s) (i.e., the CD8 HTM, 4-1BB molecule and/or cytoplasmic domain of CD32) encoded by the degenerate nucleotide sequence maintains the desired activity or function. The term “degenerate nucleotide sequence” (also known as “nucleotide degeneracy”) denotes a sequence of nucleotides that includes one or more degenerate codons (as compared to a reference polynucleotide molecule that encodes a polypeptide). Degenerate codons contain different triplets of nucleotides, but encode the same amino acid residue (e.g., GAU and GAC triplets each encode Asp). Accordingly, the nucleotide sequences comprising degenerate nucleotide(s) are intended to be included within the scope of the present disclosure, providing that the variations in the nucleotide sequence maintain at least 85% sequence identity to SEQ ID NO: 27, 28 and 29, such as at least 85%, 86%, 87%, 88%, 89%, 90%, 91%, 92%, 93%, 94%, 95%, 96%, 97%, 98% or 99% sequence identity to SEQ ID NO: 27, 28 and 29.

As described above, the nucleic acid may be modified to comprise one or more degenerate nucleotides as long as the protein (i.e., the 2B6 scFv) encoded thereby maintains the desired activity or function. Accordingly, the nucleotide sequences comprising degenerate nucleotide(s) are intended to be included within the scope of the present disclosure, providing that the variations in the nucleotide sequence maintain at least 85% sequence identity to SEQ ID NOs: 18-21, such as at least 85%, 86%, 87%, 88%, 89%, 90%, 91%, 92%, 93%, 94%, 95%, 96%, 97%, 98% or 99% sequence identity to SEQ ID NOs: 18-21.

The present invention also provides an expression vector including the nucleic acid described above. According to some embodiments, the expression vector is a viral vector, for example, a lentiviral vector, an adenoviral vector, a retroviral vector, an adeno-associated viral vector, or a sindbis viral vector. In some exemplary embodiments, the expression vector is a lentiviral vector.

(iii) Cells Expressing the CAR and Uses Thereof in Treating Cancers

The nucleic acid or expression vector described in Section (ii) of the present disclosure may be used to transform a cell (e.g., a T cell) thereby producing a genetically modified cell (e.g., CAT-T cell). Specifically, the nucleic acid or expression vector may be introduced into a cell, preferably an immune cell (e.g., T cell, NK cell or macrophage), via a transfection method known in the art; for example, chemical transfection (e.g., calcium phosphate transfection, liposome transfection or non-liposome transfection), or physical transfection (e.g., microinjection, electroporation or biolistic particle delivery). Alternatively, in the case when the expression vector is a viral vector (e.g., a lentiviral vector), it may be introduced into a host cell (e.g., HEK293T cell) via a transfection method to produce the virus (e.g., lentivirus), followed by infecting the cell (e.g., T cell, NK cell or macrophage) with the virus to achieve the gene expression purpose.

The thus-produced cell (e.g., CAR-T cell) is characterized by, (a) having the CAR expressed on its cell surface that allows the cell to specifically target and destroy cancer cells; and (b) producing and secreting anti-PD-1 antibody (i.e., anti-PD-1 scFv or anti-PD-1 scFv-Fc) that reduces the immunosuppression in tumor microenvironment thereby improving the anti-tumor response of the CAR-expressing cell (e.g., CAR-T cell) in solid tumors.

Accordingly, another aspect of the present disclosure pertains to a genetically modified cell (i.e., a cell expressing the CAR), and uses of the cell in the treatment of cancer.

Depending on desired purpose, the cell modified with the present nucleic acid or expression vector may be a T cell, NK cell or macrophage. According to some preferred embodiments, the genetically modified cell is a T cell (i.e., a CAR-T cell). The method of treating a cancer in a subject comprises administered to the subject an effective amount of the genetically modified cell (e.g., CAR-T cell, CAR-NK cell or CAR-macrophage) to alleviate or ameliorate the symptoms of the cancer.

According to certain embodiments, the subject is a mouse, in which about 1×10⁴ to 1×10⁸ (e.g., 1×10⁴, 1.5×10⁴, 2×10⁴, 2.5×10⁴, 3×10⁴, 3.5×10⁴, 4×10⁴, 4.5×10⁴, 5×10⁴, 5.5×10⁴, 6×10⁴, 6.5×10⁴, 7×10⁴, 7.5×10⁴, 8×10⁴, 8.5×10⁴, 9×10⁴, 9.5×10⁴, 1×10⁵, 1.5×10⁵, 2×10⁵, 2.5×10⁵, 3×10⁵, 3.5×10⁵, 4×10⁵, 4.5×10⁵, 5×10⁵, 5.5×10⁵, 6×10⁵, 6.5×10⁵, 7×10⁵, 7.5×10⁵, 8×10⁵, 8.5×10⁵, 9×10⁵, 9.5×10⁵, 1×10⁶, 1.5×10⁶, 2×10⁶, 2.5×10⁶, 3×10⁶, 3.5×10⁶, 4×10⁶, 4.5×10⁶, 5×10⁶, 5.5×10⁶, 6×10⁶, 6.5×10⁶, 7×10⁶, 7.5×10⁶, 8×10⁶, 8.5×10⁶, 9×10⁶, 9.5×10⁶, 1×10⁷, 1.5×10⁷, 2×10⁷, 2.5×10⁷, 3×10⁷, 3.5×10⁷, 4×10⁷, 4.5×10⁷, 5×10⁷, 5.5×10⁷, 6×10⁷, 6.5×10⁷, 7×10⁷, 7.5×10⁷, 8×10⁷, 8.5×10⁷, 9×10⁷, 9.5×10⁷, or 1×10⁸) of CAR-T cells are transferred to the subject. Preferably, about 1×10⁵ to 1×10⁷ of CAR-T cells are transferred to the subject. More preferably, about 5×10⁵ to 1×10⁶ of CAR-T cells are transferred to the mouse subject. In one specific example, about 6×10⁵ of CAR-T cells are sufficient to provide a protective and/or therapeutic effect in the mouse subject.

In general, 1×10⁶ to 1×10⁷ (e.g., 1×10⁶, 1.5×10⁶, 2×10⁶, 2.5×10⁶, 3×10⁶, 3.5×10⁶, 4×10⁶, 4.5×10⁶, 5×10⁶, 5.5×10⁶, 6×10⁶, 6.5×10⁶, 7×10⁶, 7.5×10⁶, 8×10⁶, 8.5×10⁶, 9×10⁶, 9.5×10⁶, or 1×10⁷) CAR-T cells/Kg body weight of the subject per transplant dose are required for human CAR-T therapy. As could be appreciated, the number of CAR-T cells transferred into the human subject may vary with clinical factors, such as age, gender, underlying diseases, treatment plan, conditioning regimen and infection. A skilled artisan or medical practitioner may adjust or optimize the transferred number of CAR-T cells in accordance with desired purposes.

The genetically modified cells may be autologous to the subject (i.e., being harvested from the subject having the cancer), allogeneic to the subject (i.e., being harvested from another subject, who is of the same species as the subject having the cancer), or xenogeneic to the subject (i.e., being harvested from a donor that is of a different species relative to the subject having the cancer). Preferably, the genetically modified cells are derived from the subject being treated/administered so as to avoid transplant rejection. In the case when the genetically modified cells are allogeneic or xenogeneic to the subject, the method further comprises the step of administering to the subject an immunosuppressive treatment prior to, concurrently with, or after the administration of genetically modified cells to suppress the immune response of the subject against the allogeneic or xenogeneic cells. The immunosuppression may be achieved by any agent and/or method known by a skilled artisan to prevent transplant rejection, for example, the administration of gamma irradiation or immunosuppressant.

Depending on desired purposes, the immunosuppressant may be a glucocorticoid (e.g., prednisone, budesonide, prednisolone, dexamethasone or hydrocortisone), janus kinase inhibitor (e.g., tofacitinib), calcineurin inhibitor (e.g, cyclosporine or tacrolimus), mTOR inhibitor (e.g., sirolimus or everolimus), inhibitor of inosine monophosphate dehydrogenase (IMDH inhibitor; e.g., azathioprine, leflunomide or mycophenolate), biologics or monoclonal antibody (e.g., abatacept, adalimumab, anakinra, certolizumab, etanercept, golimumab, infliximab, ixekizumab, natalizumab, rituximab, secukinumab, tocilizumab, ustekinumab, vedolizumab, basiliximab or daclizumab), or any agent known to suppress or reduce the immune response, such as methotrexate or mercaptopurine. A clinical practitioner or a skilled artisan may determine the type of immunosuppressant and treatment regimen in accordance with the physical conditions of the subject.

The genetically modified cell may be administered to the subject via any appropriate route, for example, intravenous, intraperitoneal, intraarterial or intratumoral injection. Preferably, the genetically modified cell is intravenously injected to the subject.

As would be appreciated, the present method can be applied to the subject, alone or in combination with additional therapies that have some beneficial effects on the prevention or treatment of cancers, for example, immunotherapy (e.g., the treatment of PD-1 inhibitor or PD-L1 inhibitor), surgery, chemotherapy and/or radiation therapy. Depending on the intended/therapeutic purpose, the present method can be applied to the subject before, during, or after the administration of the additional therapies.

Non-limiting examples of cancers treatable with the present method and/or pharmaceutical composition include, gastric cancer, lung cancer, bladder cancer, breast cancer, pancreatic cancer, renal cancer, colorectal cancer, cervical cancer, ovarian cancer, brain tumor, prostate cancer, hepatocellular carcinoma, melanoma, esophageal carcinoma, multiple myeloma, and head and neck squamous cell carcinoma.

Basically, the subject treatable by the present method and/or pharmaceutical composition is a mammal, for example, human, mouse, rat, guinea pig, hamster, monkey, swine, dog, cat, horse, sheep, goat, cow, and rabbit. Preferably, the subject is a human.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLE

### Materials and Methods

### Production of mAb 2B6

Two BALB/c mice were immunized with PD-1 extracellular domain for generation the anti-PD-1 hybridomas. After screening with enzyme-linked immunosorbent assay (ELISA), a mouse hybridoma for production anti-PD-1 antibody, clone 2B6, was identified and selected for further studies. The VH and VL domains of mouse anti-PD-1 antibody, clone 2B6, were then sequenced and subcloned into expression vectors. The specific steps for preparing the anti-PD-1 antibody, clone 2B6, were as follows: the expression vectors respectively encoding the VH and VL domains of anti-PD-1 2B6 antibody were transfected into FreeStyle™ 293 cells using polyethylenimine (PEI) as the transfection reagent, with a ratio of 1.25 micrograms of plasmid DNA per 1×10⁶ cells. After transfection, the cells were acclimated in FreeStyle™ 293 expression medium and cultured in a tissue culture flask. The culture supernatant was collected when the viable cell rate reached 90%. The collected culture supernatant was filtered through 10 micrometer and 0.2 micrometer filters to remove contaminants. The antibody was affinity-purified using Protein A, with phosphate buffered saline (PBS) as the absorption buffer and 200 mM glycine buffer (pH 2.5) as the elution buffer. The elution fractions were adjusted to a pH of around 6.0-7.0 by adding 50 mM Tris buffer (pH 9.0). The antibody solution was replaced with PBS using a dialysis membrane (10,000 MW cut and filter-sterilized through a 0.22 micrometer membrane filter. The concentration of the purified antibody was determined by measuring the absorbance at 280 nm and converting the measured value based on the conversion factor that an optical density of 1.45 equals 1 mg/ml.

The thus-produced mAb was designated as “mAb 2B6”, in which the VH and VL sequences are summarized in Table 1.

TABLE 1
VH and VL sequences of mAb 2B6

| | | SEQ ID |
|---|---|---|
| Name | Amino acid sequence | NO |
| mAb 2B6 | EVKLVESGGGLVKPGGSLKLSCAAAGFTFSSYTMSWVRQTPAKR | 7 |
| VH | LEWVATISGGGANIYYPDSVKGRFTISRDNARNTLYLQMSSLRS | |
| | EDTAMYYCVSPYYAIDFWGQGTSVTVSS | |
| mAb 2B6 | DTVMTQSDKFMSTSVGDRVSITCKASQDVGSAVAWYQQKPGQSP | 8 |
| VL | KLLIYWASTRHTGVPDRFTGSGSGTDFTLTISNVQSEDLADYFC | |
| | QQYSTYTWTFGGGTKLEIK | |

The CDR sequences (including CDR-H1, CDR-H2 and CDR-H3 of VH domain, and CDR-L1, CDR-L2 and CDR-L3 of VL domain) were marked in bold, in sequence.

### Humanization of mAb 2B6

### (A) Selection of Human Variable Region Framework Sequences

Mouse mAb may induce potent immunogenicity and anti-drug antibody in human patients. Therefore, humanization of mouse mAb is an essential and critical step for further drug development.

For the preparation of humanized 2B6 4D5 (2B6-HdHd), the human acceptor framework was selected from a framework that has been validated in the clinical trial study. Human heavy and light chain framework sequences in the VH subgroup III (IGHV3) and VL κ subgroup I (IGKV1) have been validated in the clinic and been used in many humanized antibodies with success.

For the preparation of humanized 2B6 IMGT VH (2B6-HuHd), human germ-line VH sequences with the highest degree of homology with the mAb 2B6 framework regions were identified from the IMGT database (the International Immunogenetics Information System®). The homology searches may be performed with BLAST or similar methods. The mAb 2B6 variable region sequences were used as query sequences. The research led to the identification of the human germline gene IGH3-23*04 (VH), as the sequences most homologous to the corresponding heavy chain framework sequences in mAb 2B6.

These two pairs of heavy chain sequences (human 4D5 and human IMGT) and light chain (human 4D5) were used as templates for the construction of humanized antibodies against human PD1.

### (B) Back Mutation

Grafting of CDR onto frameworks resulted in variable domains (VH and VL) from different sources. Such chimeric domains may not have the optimal sequences. Therefore, the affinities of the antibodies may not be the best. To improve the binding affinity, some amino acids may be mutated back to the other species. These critical amino acid residues sometimes influence antibody bindings that are located the antibody upper core region and the interface area. According to the principles described above, three back mutations were respectively introduced into humanized 2B6 HdB1 (VH) and 2B6 HuB1 (VH) (respectively at positions 73, 93 and 94 of the framework regions).

The amino acid sequences of the thus-produced humanized VH and VL domains were summarized in Table 2.

TABLE 2
Amino acid sequences of humanized VH and VL domains

| | | SEQ ID |
|---|---|---|
| Name | Amino acid sequence | NO |
| 2B6 Hd | EVQLVESGGGLVQPGGSLRLSCKASGFTFSSYTMSWVRQAPGKG | 12 |
| VH | LEWVATISGGGANIYYPDSVKGRFTISRDDSKNTLYLQMNSLRA | |
| | EDTAVYYCARPYYAIDFWGQGTLVTVSS | |
| 2B6 HdB1 | EVQLVESGGGLVQPGGSLRLSCKASGFTFSSYTMSWVRQAPGKG | 13 |
| VH | LEWVATISGGGANIYYPDSVKGRFTISRDNSKNTLYLQMNSLRA | |
| | EDTAVYYCVSPYYAIDFWGQGTLVTVSS | |
| 2B6 HuB1 | EVQLVESGGGLVQPGGSLRLSCAASGFTFSSYTMSWVRQAPGKG | 14 |
| VH | LEWVATISGGGANIYYPDSVKGRFTISRDNSKNTLYLQMNSLRA | |
| | EDTAVYYCVSPYYAIDFWGQGTLVTVSS | |
| 2B6 Hd | DIQMTQSPSSLSASVGDRVTITCKASQDVGSAVAWYQQKPGKAP | 15 |
| VL | KLLIYWASTRHTGVPSRFSGSGSGTDFTLTISSLQPEDFATYYC | |
| | QQYSTYTWTFGQGTKVEIK | |

### Expression of Humanized Antibodies

To confirm the change in affinity after humanization of the mouse antibody, the variable regions of humanized light chain and humanized heavy chains were directly generated by the nucleotide synthesis method, respectively. The mouse or humanized variable regions were constructed into human chimera antibody mammalian expression vector, and that were introduced into host cells to prepare recombinant antibody-expressing cells. As the host cells for expression, the FreeStyle™ 293 or Expi™ 293 cells were used. The vectors were introduced into the host cells by polyethylimine (PEI), in which about 1.25 microgram of the antibody expression vector was introduced into 1×10⁶ cells. Transient expressed 30 ml culture supernatant containing human IgG antibody was prepared by the method described below. The antibody producing cells were acclimated in a FreeStyle™ 293 expression medium. The cells were cultured in 135 rpm orbital shaking culture flask in 8% CO₂ at 37° C., and the supernatants were collected at day 6 after transfection. The collected supernatant was filtered through 0.2 micrometer filters. The culture supernatant containing the antibody was affinity-purified using Protein A, PBS as an absorption buffer, and 200 mM glycine buffer (pH2.5) as an elution buffer. The elution fractions were adjusted to around pH 6.0-7.0 by adding 50 mM Tris buffer (pH9.0). The prepared antibody solution was replaced with PBS using a dialysis membrane (10,000 MW cut) and filter-sterilized through a membrane filter having a pore size of 0.22 micrometer to yield the purified antibody. The concentration of the purified antibody was determined by measuring the absorbance at 280 nm and converting the measured value based on 1.45 optimal density equaling 1 mg/ml (Table 3).

TABLE 3
Expression and yield of specified antibody

| | | | | OD280 | | |
|---|---|---|---|---|---|---|
| | | Heavy | Light | Conc. | Vol. | Yield |
| | Target | chain | chain | (ug/ul) | (ul) | (ug) |
| M-M | PD-1 | M | M | 2.6 | 100 | 260 |
| Hd-Hd | PD-1 | Hd | Hd | 3.8 | 100 | 380 |
| HdB1-Hd | PD-1 | HdB1 | Hd | 2.6 | 100 | 260 |
| HuB1-Hd | PD-1 | HuB1 | Hd | 2.6 | 150 | 390 |

Conversion of Full IgG into scFv-Fc Format

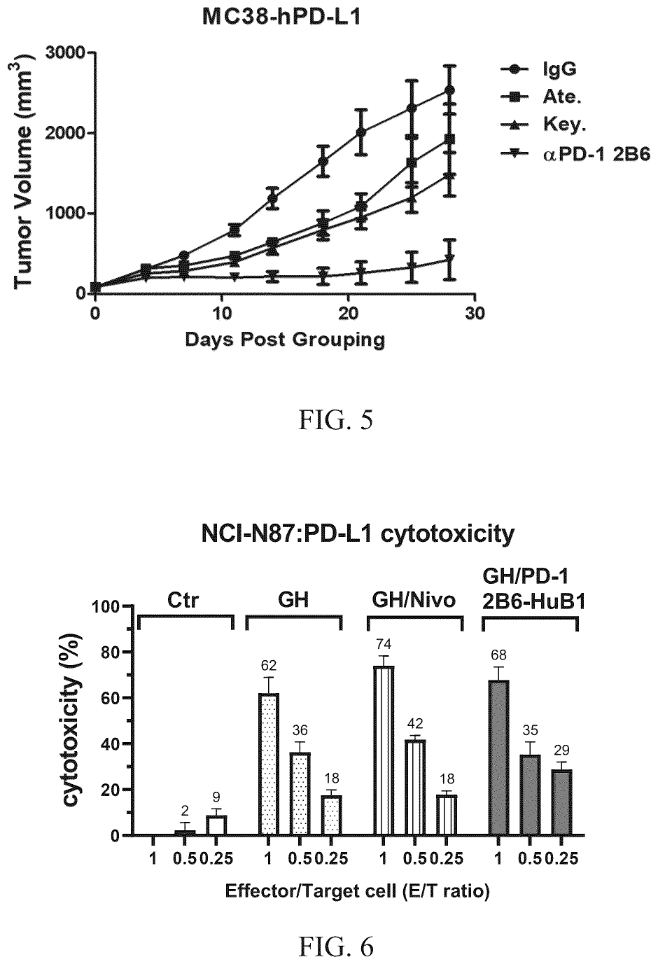

The heavy chain variable region (VH) and light chain variable region (VL) of humanized 2B6 antibody, HuB1 Hd, was cloned as scFv (VH-linker (Gly4Ser)3-VL)-Fc format by PCR based method. The recombinant humanized 2B6 HuB1-scFv-Fc (scFv format from 2B6 (HuB1 Hd)), Pembro-scFv-Fc (scFv format form Pembrolizumab) and Nivo-scFv-Fc (scFv format from Nivolumab) antibodies were respectively expressed in FreeStyle™ 293 cells (FIG. 5A). All purified scFv-Fc antibodies were quantified by OD260/280 and amount of each antibodies were listed in Table 4. The data indicated that the amount of 2B6 HuB1-scFv-Fc was significantly superior to those of Pembro-scFv-Fc and Nivo-scFv-Fc.

TABLE 4
Expression and yield of specified antibody

| | | | OD280 | | |
|---|---|---|---|---|---|
| | | | Conc. | Vol. | Yield |
| Name | Format | Original Ab | (ug/ul) | (ul) | (ug) |
| 286 HuB1-scFv-Fc | scFv-Fc | 2B6 (HuB1Hd) | 7.1 | 200 | 1420 |
| Pembro-scFv-Fc | scFv-Fc | Pembrolizumab | 0.08 | 200 | 16 |
| Nivo-scFv-Fc | scFv-Fc | Nivolumab | 1.4 | 200 | 280 |

Determination of Binding Affinity of Humanized Antibodies and scFv-Fc Antibodies to PD1 by Using Enzyme-Linked Immunosorbent Assay (ELISA)

ELISA plates were coated with PD1-hFc (1 ug/ml) and blocked with blocking buffer (5% milk dissolved in PBS). The antibodies from 66.67 nM to 2×10⁴ nM dilute (4× fold dilution) were then added to the plates. The binding affinity of tested antibody to PD1-hFc was determined via adding goat anti-human KAPPA HRP IgG (1:3,000 dilution), and binding curve and KD were determined by software using one site-specific binding for nonlinear fit methods.

### CAR Plasmid Construction

A human immunodeficiency virus (HIV)-1-based lentiviral expression vector (pLVX-EF1a-IRES) was used in this study. The DNA fragments respectively encoding Globo H scFv, CD8 hinge and transmembrane domains, co-stimulatory molecule 4-1BB, CD3ζ domain, T2A, signal peptide (SEQ ID NO: 30), humanized 2B6 HuB1, and human IgG4 Fc domain were synthesized and assembled into a CAR gene cassette. The assembled cassette was inserted into pLVX-EF1a-IRES vector via EcoRI and BamHI restriction enzyme sites. The assembled cassette was inserted into pLVX-EF1a-IRES vector via EcoRI and BamHI restriction enzyme sites.

The thus-produced plasmid was designated as “Globo H/PD-1^{2B6} scFv-Fc CAR plasmid” (FIG. 2).

Three plasmids serving as positive controls were provided in the present study, including, (1) the plasmid encoding Globo H CAR (designated as “Globo H CAR plasmid”); (2) the plasmid encoding Globo H CAR and Pembrolizumab scFv-Fc (designated as “Globo H/Pembro scFv-Fc CAR plasmid”); and (3) the plasmid encoding Globo H CAR and Nivolumab scFv-Fc (designated as “Globo H/Nivo scFv-Fc CAR plasmid”) (FIG. 2).

The Globo H/PD-1^{2B6} scFv-Fc CAR plasmid, Globo H/Pembro scFv-Fc CAR plasmid, and Globo H/Nivo scFv-Fc CAR plasmid were respectively expressed in FreeStyle™ 293 cells. All purified scFv-Fc antibodies were quantified by OD260/280. Also, the binding affinity of the thus-produced antibodies towards PD-1 was determined in accordance with the procedures described above.

On the other hand, the humanized 2B6 HuB1 was also fused with B7-H3 scFv (SEQ ID NO: 31). The thus-produced plasmid was designated as “B7-H3/PD-1^{2B6} scFv-Fc CAR plasmid”, which comprised the DNA fragments respectively encoding B7-H3 scFv (SEQ ID NO: 32), CD8 hinge and transmembrane domains, co-stimulatory molecule 4-1BB, CD33 domain, T2A, signal peptide, humanized 2B6 HuB1, and human IgG4 Fc domain. In the study, the plasmid encoding B7-H3 CAR (designated as “B7-H3 CAR plasmid”) served as a positive control.

### Lentivirus Production

1×10⁷ 293T cells were seeded in 15-cm dish. Before transfection, culture medium was replaced by fresh DMEM medium containing 10% fetal bovine serum (FBS). Three plasmids, including pMD.G (6 ug), R8.91 (15 ug) and transfer plasmid (20 ug), were mixed with transfection reagent polycation polyethylenimine (PEI) at a volume ratio of 1:2.5, followed by incubating at room temperature for 20 minutes. Then, the mixture was added into 293T cells. 16 hours later, culture medium was changed with DMEM medium with 2% FBS. The culture supernatant containing viral particles were harvested at 48 and 72 hours post-transfection, and then mixed with concentration reagent overnight, followed by centrifuging the mixture at 1,500×g for 30 minutes at 4° C. The lentiviral particles were resuspended in media, and the viral titer was determinant by Jurkat cells infection and flow cytometer.

The thus-produced lentiviruses were respectively designated as “Globo H/PD-12B6 scFv-Fc CAR virus” and “B7-H3/PD-12B6 scFv-Fc CAR virus”.

Four lentiviruses serving as positive controls were provided in the present study, including, (1) the lentivirus carrying the Globo H CAR plasmid (designated as “Globo H CAR virus”); (2) the lentivirus carrying the Globo H/Pembro scFv-Fc CAR plasmid (designated as “Globo H/Pembro scFv-Fc CAR virus”); (3) the lentivirus carrying the Globo H/Nivo scFv-Fc CAR plasmid (designated as “Globo H/Nivo scFv-Fc CAR virus”); and (4) the lentivirus carrying the B7-H3 CAR plasmid (designated as “B7-H3 CAR virus”).

### CART Cells Preparation

### (i) Isolation of Peripheral Blood Mononuclear Cells (PBMCs)

The blood sample (10 ml) isolated from healthy donor was diluted with 1× phosphate buffered saline (PBS; 10 mL) or balanced salt buffer. Lymphoprep media (15 ml) were added to the centrifuge tube, followed by carefully layering the diluted blood sample (total 20 ml) onto the Lymphoprep media solution, and then centrifuging at 800×g for 20 minutes at 15° C.-20° C. with brake off. The upper layer containing plasma and platelets was discarded using a sterile pipette. The mononuclear cell layer undisturbed at the interface was transferred to a sterile centrifuge tube (8 mL), and mixed with at least 3 volumes (about 25 ml) of 1×PBS. After centrifuging at 500×g for 10 minutes at 20° C., the supernatant was discarded, and 20 mL of 1×PBS were added to the mononuclear cells in the centrifuge tube. The tube was centrifuged at 500×g for another 10 minutes at 20° C., and then washed with 1×PBS again. The cell pellet was resuspended in media appropriate for the cell number determination.

### (ii) Isolation of Pan T Cells

After determining the cell number, the tube was centrifuged at 300×g for 10 minutes. The thus-obtained cell pellet was resuspended in buffer, and mixed with CD3 MicroBeads. The mixture was incubated for 15 minutes at 4-8° C. After washing the cells with 1-2 mL of buffer and centrifuging at 300×g for 10 minutes, the cells were resuspended in buffer and then added to the column in the magnetic field of Separator QuadroMACS™. Unlabeled cells which pass through were collected, and the column was washed with 3 mL of buffer three times. The column was removed from the separator and placed on a suitable collection tube. 5 mL of 1×PBS were added to the column, the fraction containing the magnetically labeled cells was immediately flushed out by firmly applying the plunger supplied with the column. The flowthrough was collected and the T cell number was determined.

(iii) T Cell Activation

The Dynabeads® Human T-Activator CD3/CD28 were added to the purified T cell (Beads:Cells=2:1). Culture medium were changed every 2 days.

### (iv) Viral Transduction

Primary T cells were seeded in a 6-well plate (1.8×10⁶ cells/well). Lentivirus carrying CAR-encoding nucleic acid was added to the cells (MOI=1; MOI: multiplicity of infection), followed by centrifuging at 800×g for 90 minutes, and incubating at 37° C. overnight. Then, 2 ml medium containing IL-2 (125 U/ml) were added, and the expression of GFP (Fab) was detected by flow cytometry on Day 4, Day 7 and Day 10.

### (v) CAR-T Cell Expansion

The cell density was adjusted to 5×10⁵/mL with medium. On day 4, the medium were changed, and the cells were cultured with 30 mL Bioreactor (starting with 9×10⁶ total cell for expansion; 120 rpm). On day 7, the medium were changed, and the cells were cultured with 100 mL Bioreactor (starting with 3×10⁷ total cell for expansion; 90 rpm). The CAR-T cells were harvested on day 10.

The thus-produced CAR-T cells were respectively designated as “Globo H/PD-1^{2B6} scFv-Fc CAR-T cell” and “B7-H3/PD-1^{2B6} scFv-Fc CAR-T cell”.

The T cells respectively transduced with the Globo H CAR virus (designated as “Globo H CAR-T cell”), the Globo H/Pembro scFv-Fc CAR virus (designated as “Globo H/Pembro scFv-Fc CAR-T cell”), the Globo H CAR/Nivo scFv-Fc CAR virus (designated as “Globo H CAR/Nivo scFv-Fc CAR-T cell), and the B7-H3 CAR virus (designated as “B7-H3 CAR-T cell”) served as positive controls in the present study.

### Cytotoxicity Assay

Target cells were seeded in a 96-well plate at a concentration of 2×10⁴ cells/well in triplicates. Subsequently, CAR-T cells (effector cells) were added at different effector-to target (E:T) ratios, including 1:1, 0.5:1, and 0.25:1 for NCI-N87/PD-L1. The cells were incubated at 37° C. for 24 hours. The cells were washed with RPMI1640 culture medium twice, and cell counting kit (CCK-8) was used to determine the number of viable cells in the cytotoxicity assay.

### Animal Study

The NCI-N87 with PD-L1 highly expression cells (N87/PD-L1) used for implantation were harvested during log phase growth and re-suspended in PBS with 50% Matrigel® Basement Membrane Matrix to a concentration containing 3×10⁷ cells/mL. N87/PD-L1 tumor cells (3×10⁶ cells) in a dose volume of 0.1 mL were subcutaneously (SC) injected to the right front flank of mice for tumor growth. Ten days post tumor cell inoculation, when the mean tumor volume (MTV) reached approximately 117 mm³, tumor-bearing mice were randomly divided to 7 groups, in which each group consisted of 4 mice, and administrated with CAR-T cells or vehicle solution on the same study day. Globo H, Globo H/PD-1 Nivo scFv-Fc, or Globo H/PD-1^{2B6} scFv-Fc CAR-T cell suspensions (6×10⁵ CAR-T cells in a dose volume of 0.1 mL) were immediately IV injection for a single dose. Tumors were measured three times per week using digimatic calipers, and the tumor volume was expressed in mm³ using the formula: TV=(w²×l)/2; where w=width and l=length in diameter (mm) of the tumor. Tumor growth inhibition (TGI) rate was calculated using the following formula: % TGI=[1−(T/C)]×100%; where T and C represent the MTV of the treatment group and the vehicle control group, respectively.

The Cal-27 cells used for implantation were harvested during log phase growth and re-suspended in PBS with 50% Matrigel® Basement Membrane Matrix to a concentration containing 1×10⁷ cells/mL. Cal-27 tumor cells (1×10⁶ cells) in a dose volume of 0.1 mL were subcutaneously (SC) injected to the right front flank of mice for tumor growth. Ten days post tumor cell inoculation, when the mean tumor volume (MTV) reached approximately 141 mm³, tumor-bearing mice were randomly divided to 3 groups, in which each group consisted of 4 mice, and administrated with CAR-T cells or vehicle solution on the same study day. B7-H3 or B7-H3/PD-1^{2B6} scFv-Fc CAR-T cell suspensions (6×10⁵ CAR-T cells in a dose volume of 0.1 mL) were immediately IV injection for a single dose. Tumors were measured three times per week using digimatic calipers, and the tumor volume was expressed in mm³ using the formula: TV=(w²×l)/2; where w=width and l=length in diameter (mm) of the tumor. Tumor growth inhibition (TGI) rate was calculated using the following formula: % TGI=[1−(T/C)]×100%; where T and C represent the MTV of the treatment group and the vehicle control group, respectively.

### Example 1: Binding Affinity of Chimeric Anti-Human PD-1 2B6 Antibody to PD-1 Determined by ELISA

Antibodies of the invention were evaluated for specific binding to PD-1 using an enzyme-linked immunosorbent assay (ELISA). Briefly, recombinant PD-1 protein was immobilized on a 96-well ELISA plate at 0.1 μg/well. The chimeric anti-human PD-1 2B6 antibody was serially diluted and incubated with the coated PD-1, followed by detection with a horseradish peroxidase (HRP)-conjugated goat anti-mouse IgG secondary antibody. The binding was visualized using 3,3′,5,5′-tetramethylbenzidine (TMB) as a substrate, and the optical density (OD) was measured at 405 nm to assess the binding activity.

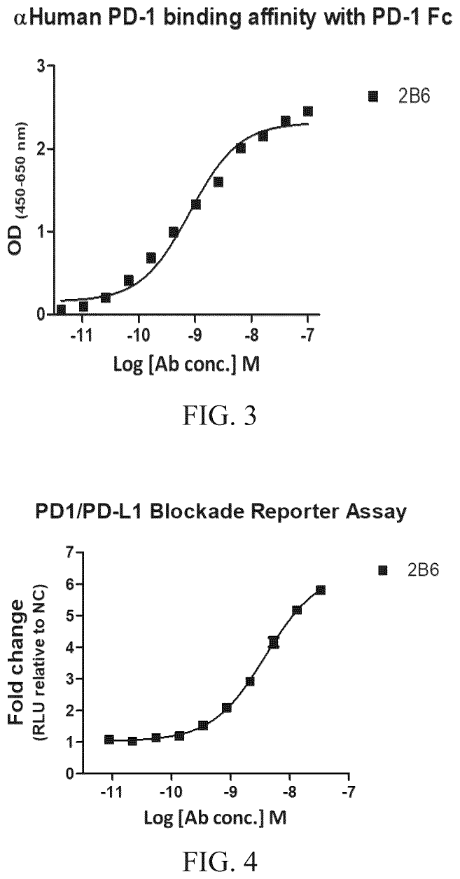

As shown in FIG. 3, the chimeric anti-human PD-1 2B6 antibody exhibited specific and high-affinity binding to PD-1, with a dissociation constant (K_{D}) of 8.09×10⁻¹⁰ M, indicating a tight interaction between the antibody and its target.

### Example 2: Dose-Dependent Blockade of PD-1/PD-L1 Interaction by Chimeric Anti-Human PD-1 2B6 Antibody in a Bioluminescent Cell-Based Assay

To evaluate the functional activity of the antibodies of the invention in blocking PD-1/PD-L1 interactions, a bioluminescent cell-based assay was conducted using the Promega PD-1/PD-L1 Blockade Bioassay kit (Promega, Madison, WI, USA). The assay utilizes two genetically engineered cell lines: PD-1 Effector Cells (Jurkat T cells expressing human PD-1 and a luciferase reporter driven by an NFAT response element, NFAT-RE) and PD-L1 aAPC/CHO-K1 Cells (CHO-K1 cells expressing human PD-L1 and an engineered cell surface protein that activates cognate TCRs in an antigen-independent manner).

When co-cultured, the PD-1/PD-L1 interaction would inhibit TCR signaling and NFAT-RE-mediated luminescence. The addition of antibodies that blocked PD-1/PD-L1 interactions (e.g., the present chimeric anti-human PD-1 2B6 antibody), resulted in the release of this inhibitory signal, leading to TCR activation and a dose-dependent increase in NFAT-RE-mediated luminescence. The bioluminescent signal was detected and quantified using the Bio-Glo™ Luciferase Assay System and a luminometer (GloMax® Discover System, Promega, Madison, WI, USA).

As shown in FIG. 4, the chimeric anti-human PD-1 2B6 antibody demonstrated potent and specific blockade of PD-1/PD-L1 interactions, with an EC₅₀ of 3.88×10⁻⁹M (approximately 3.88 nM). Other antibodies of the present disclosure exhibited similar inhibitory activities (data not shown). These results confirmed that the antibodies of the present disclosure could effectively relieve immune suppression mediated by PD-1/PD-L1 interactions in a cellular context, suggesting their potential therapeutic utility in treating diseases driven by immune suppression or exhaustion, such as cancers.

### Example 3: In Vivo Anti-Tumor Efficacy of Chimeric Anti-PD-1 2B6 Antibody in MC38-hPD-L1 Syngeneic Tumor Model

A murine syngeneic model established in accordance with the procedures set forth in “Material and methods” section was employed to evaluate the anti-cancer effect of the chimeric anti-human PD-1 2B6 antibody of the present disclosure. Briefly, B-hPD-1/hPD-L1 mice were subcutaneously inoculated with MC38-hPD-L1 tumor cells (5×10⁵) suspended in 0.1 mL PBS into the right front flank to establish tumor growth. Tumor-bearing mice were randomly assigned to seven study groups (G1: 6 mice; G2-G4: 8 mice each) when the mean tumor volume reached 75±25 mm³. The treatment groups received intraperitoneal administrations of test articles at 5 mg/kg, including murine IgG (control), atezolizumab (Ate.), keytruda (Key.), and the chimeric anti-PD-1 2B6 antibody, administered twice weekly for a total of six doses. Tumor volumes and body weights were measured twice per week. The study was terminated on Day 28 (7 days post final dosing). At endpoint, tumors were excised, weighed, and photographed. Results are depicted in FIG. 5.

It was found the present 2B6 antibody significantly suppressed tumor growth, as compared to that of control, and showed comparable or superior efficacy to commercially available anti-PD-1/PD-L1 antibodies, demonstrating its potential as a novel immunotherapeutic candidate.

### Example 4: Binding Affinities of Humanized 2B6 Antibodies to PD-1

The binding affinity of the present antibodies listed in Tables 3 and 4 towards PD-1 was determined by ELISA as described in “Materials and Methods” of the present disclosure.

Compared to parental mouse clone 2B6 (M-M), which had a binding affinity of about 4.95×10⁻¹⁰ M, the binding affinities of humanized antibody 2B6 HdB1 Hd and 2B6 HuB1 Hd to PD-1 were respectively 3.68×10⁻¹⁰ and 4.03×10⁻¹⁰ M. In comparison to Hd (VH), each of HdB1 and HuB1 (VH) contains three beneficial mutations in its heavy chain framework region and exhibited greater binding affinity towards PD-1 (Table 5), suggesting that these residues in the framework regions contribute indirectly to the binding of PD-1.

TABLE 5
The binding affinity of the present antibodies

| | Name of the antibody | | | |
|---|---|---|---|---|
| | M-M | Hd-Hd | HdB1 Hd | HuB1 Hd |
| K_{D} (M) | 4.95 × 10⁻¹⁰ | 1.79 × 10⁻⁷ | 3.68 × 10⁻¹⁰ | 4.03 × 10⁻¹⁰ |

The KD values of 2B6 HuB1-scFv-Fc, Pembro-scFv-Fc and Nivo-scFv-Fc antibodies were respectively 2.58×10⁻⁹, 2.79×10⁻⁹ and 6.68×10⁻⁹ M (Table 6). The binding affinity of 2B6 HuB1-scFv-Fc was significantly superior to that of Nivo-scFv-Fc and similar to that of Pembro-scFv-Fc.

TABLE 6
The binding affinity of the present antibodies

| | Name of the antibody | | |
|---|---|---|---|
| | 2B6 HuB1-scFv-Fc | Pembro-scFv-Fc | Nivo-ScFv-Fc |
| K_{D} (M) | 2.58 × 10⁻⁹ | 2.79 × 10⁻⁹ | 6.68 × 10⁻⁹ |

### Example 5: Expression of the Present CAR Construct

To evaluate the expression of the present CAR construct, the plasmid encoding the CAR molecule was transfected into 293T cells. The protein was harvested from the supernatant of the cultured cells, followed by purifying and quantifying via measuring OD260/280. On the other hand, the binding affinity of the purified protein towards PD-1 was determined by ELISA. The data is summarized in Table 7.

TABLE 7
Expression of specified CAR construct

| | Name of the antibody | | |
|---|---|---|---|
| | Pembro-scFv-Fc | Nivo-ScFv-Fc | 2B6 HuB1-scFv-F |
| K_{D} (M) | 4.62 × 10⁻⁹ | 5.21 × 10⁻⁹ | 1.75 × 10⁻⁹ |
| Yield (μg) | 47.6 | 61.7 | 124 |

The data of Table 7 demonstrated that the present Globo H/PD-1^{2B6} scFv-Fc CAR could be highly expressed by 293T cells and the binding affinity of 2B6 HuB1-scFv-Fc was significantly superior to that of Nivo-scFv-Fc and of Pembro-scFv-Fc towards PD-1.

### Example 6: Anti-Tumor Effect of the Present CAR-T Cell In Vitro

The cytotoxic activity of the present Globo H/PD-1^{2B6} scFv-Fc CAR-T cell was determined by co-incubating the CAR-T cell with cancer cells at different effector-to target (E:T) ratios as described in “Materials and Methods”. The data of FIG. 6 demonstrated that the present Globo H/PD-1^{2B6} scFv-Fc CAR-T cell exhibited cytotoxic activity to cancer cells in a dose-dependent manner (FIG. 6).

### Example 7: Anti-Tumor Effect of the Present CAR-T Cell

The N87/PD-L1 gastric tumor model and Cal-27 tongue squamous cell carcinoma model were used in the example to evaluate the therapeutic effect of the present CAR-T cell on cancers. As described in “Materials and Methods” of the present disclosure, single dose of CAR-T cell (6×10⁵ cells) was infused into the N87-bearing or Cal-27 mice, and the tumor volumes were monitored every two or three days.

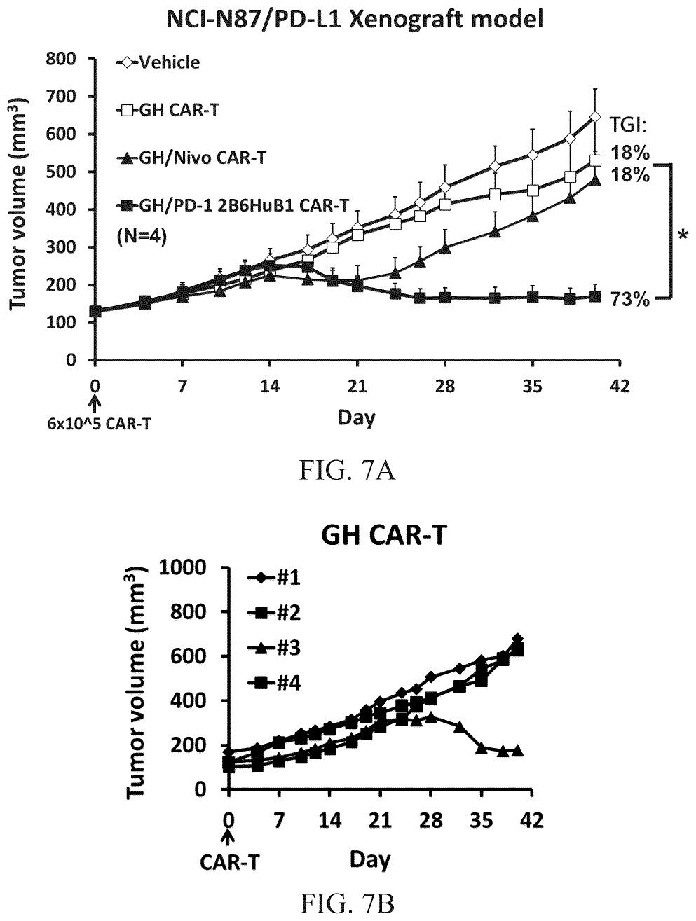

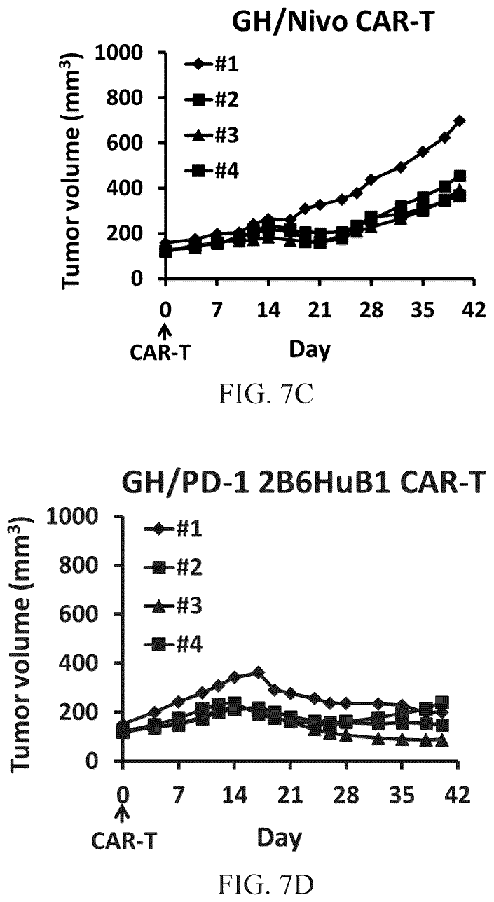

Compared to the control groups, the administration of the present CAR-T cell significantly inhibited the tumor growth in N87/PD-L1 tumor model, in which the tumor growth inhibition (TGI) rate was about 73% (FIG. 7A). Of note, the anti-tumor effect of the present CAR-T cell was obviously greater than those of the control groups, including Globo H CAR-T cell (GH CAR-T) and Globo H CAR/Nivo scFv-Fc CAR-T cell (GH/Nivo CAR-T) (FIGS. 7A-7D).

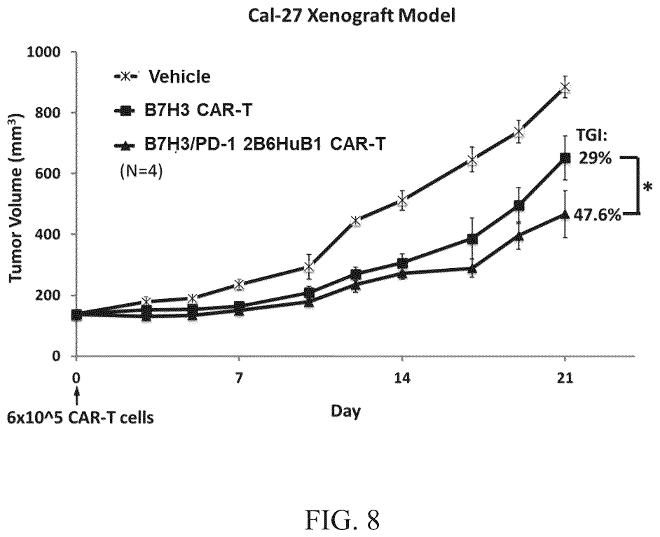

Compared to control groups, administration of the present CAR-T cell significantly inhibited the tumor growth in Cal-27 tumor model, in which the tumor growth inhibition (TGI) rate was about 47.6% (FIG. 8). Of note, the anti-tumor effect of the present B7H3/PD-1 2B6HuB1 CAR-T cell was obviously greater than those of the control groups, including, Vehicle and B7H3 CAR-T (FIG. 8).

In conclusion, the present disclosure provides a novel anti-PD-1 antibody, 2B6; and a CAR-T cell comprising the antibody 2B6. According to the examples of the present disclosure, the CAR-T cell (i.e., Globo H/PD-1^{2B6} scFv-Fc CAR-T cell) exhibited a binding affinity to PD-1 and a cytotoxic activity towards cancer cells. The present CAR-T cell is useful in producing and secreting anti-PD-1 scFv-Fc that reduces the immunosuppression in tumor microenvironment, thereby improving the anti-tumor response of the CAR-T cell in solid tumors.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A recombinant antibody comprising a heavy chain variable (VH) domain and a light chain variable (VL) domain, wherein the VH domain comprises a first heavy chain complementarity determining region (CDR-H1), a second heavy chain CDR (CDR-H2) and a third heavy chain CDR (CDR-H3), and the VL domain comprises a first light chain CDR (CDR-L1), a second light chain CDR (CDR-L2) and a third light chain CDR (CDR-L3), wherein
the CDR-H1, CDR-H2, and CDR-H3 respectively comprise the amino acid sequences of “GFTFSSYTMS” (SEQ ID NO: 1), “TISGGGANIYYPDSVKG” (SEQ ID NO: 2), and “PYYAIDF” (SEQ ID NO: 3); and
the CDR-L1, CDR-L2, and CDR-L3 respectively comprise the amino acid sequences of “KASQDVGSAVA” (SEQ ID NO: 4), “WASTRHT” (SEQ ID NO: 5), and “QQYSTYTWT” (SEQ ID NO: 6).

2. The recombinant antibody of claim 1, wherein the VH domain and VL domain respectively comprise the amino acid sequences at least 85% identical to SEQ ID NOs: 7 and 8.

3. The recombinant antibody of claim 2, wherein
the VH domain comprises the amino acid sequence of SEQ ID NO: 7, 12, 13 or 14; and
the VL domain comprises the amino acid sequence of SEQ ID NO: 8 or 15.

4. The recombinant antibody of claim 1, wherein the recombinant antibody is secreted by an immune cell or a stem cell.

5. The recombinant antibody of claim 4, wherein the immune cell is a T-cell.

6. An isolated cell configure to express a chimeric antigen receptor (CAR) and the recombinant antibody of claim 1, wherein the isolated cell is transformed by a nucleic acid comprising in sequence, from 5′-end to 3′-end,
a first coding sequence encoding a first single-chain variable fragment (scFv) specific to an antigen;
a second coding sequence encoding a hinge and transmembrane (HTM) domain of a first protein;
a third coding sequence encoding a co-stimulatory molecule;
a fourth coding sequence encoding a cytoplasmic domain of a second protein;
an internal ribosomal entry site (IRES) or a linker sequence encoding a 2A peptide; and
a fifth coding sequence encoding the recombinant antibody of claim 1;
the first to fourth coding sequences collectively encode the CAR, which is disposed on the membrane of the isolated cell after expression; and
the recombinant antibody of claim 1 is secreted out from the isolated cell after expression.

7. The isolated cell of claim 6, wherein
the antigen is a tumor-associated antigen (TAA);
the first and second proteins are respectively cluster of differentiation 8 (CD8) and CD3 zeta chain (CD3ζ); and
the co-stimulatory molecule is 4-1BB.

8. The isolated cell of claim 7, wherein the VH and VL domains of the recombinant antibody of claim 1 respectively comprise the amino acid sequences at least 85% identical to SEQ ID NOs: 7 and 8.

9. The isolated cell of claim 8, wherein
the VH domain comprises the amino acid sequence of SEQ ID NO: 7, 12, 13 or 14; and
the VL domain comprises the amino acid sequence of SEQ ID NO: 8 or 15.

10. The isolated cell of claim 7, wherein
the HTM domain of the CD8 comprises the amino acid sequence of SEQ ID NO: 9;
the 4-1BB co-stimulatory molecule comprises the amino acid sequence of SEQ ID NO: 10; and
the cytoplasmic domain of the CD35 comprises the amino acid sequence of SEQ ID NO: 11.

11. The isolated cell of claim 6, wherein the recombinant antibody of claim 1 further comprises a fragment crystallizable region (Fc region) of an immunoglobulin disposed at its C-terminus.

12. The isolated cell of claim 11, wherein the immunoglobulin is immunoglobulin G (IgG).

13. The isolated cell of claim 6, wherein the isolated cell is an immune cell or a stem cell.

14. A method of treating cancer in a subject comprising administering to the subject an effective amount of the isolated cell of claim 6 to alleviate or ameliorate symptoms of the cancer.

15. The method of claim 14, wherein the cancer is gastric cancer, lung cancer, bladder cancer, breast cancer, pancreatic cancer, renal cancer, colorectal cancer, cervical cancer, ovarian cancer, brain tumor, prostate cancer, hepatocellular carcinoma, melanoma, esophageal carcinoma, multiple myeloma, or head and neck squamous cell carcinoma.

16. The method of claim 14, wherein the isolated cell of claim 6 is a T cell, a natural killer (NK) cell, a macrophage or a stem cell.

17. The method of claim 14, wherein the subject is a human.
